(19) United States
(12) Patent Application Publication
(10) Pub. No.: US 2023/0198309 A1
(43) Pub. Date: Jun. 22, 2023

(54) MAGNETIC RESONANCE LOCAL COIL FOR PERCUTANEOUS MRT-GUIDED NEEDLE INTERVENTION

(71) Applicant: Siemens Healthcare GmbH, Erlangen (DE)

(72) Inventors: Axel Joachim Krafft, Hemhofen (DE); Matthias Malzacher, Nürnberg (DE); Martino Leghissa, Wiesenthau (DE); Florian Maier, Buckenhof (DE)

(21) Appl. No.: 18/083,472

(22) Filed: Dec. 16, 2022

(30) Foreign Application Priority Data
Dec. 17, 2021 (DE) 10 2021 214 562.0

(51) Int. Cl. H02J 50/12; H01F 38/14

(52) U.S. Cl. CPC H02J 50/12; H01F 38/14

(57) ABSTRACT

A local coil for percutaneous MRT-guided minimally invasive intervention is provided. The local coil has a central antenna coil having an opening for passing through an instrument. A first plurality of first peripheral antenna coils surround the central antenna coil on an outer circumference. The local coil is configured to be arranged flat on a body surface of a patient.

This application claims the benefit of German Patent Application No. DE 10 2021 214 562.0, filed on Dec. 17, 2021, which is hereby incorporated by reference in its entirety.

### BACKGROUND

The present embodiments relate to a local coil for percutaneous magnetic resonance tomography (MRT)-guided minimally invasive intervention.

Magnetic resonance tomography systems are imaging apparatuses that, in order to image an examination subject, align nuclear spins of the examination subject using a strong external magnetic field and excite the nuclear spins into precession around the alignment using an alternating magnetic field. The precession or return of the spins from the excited state into a state having lower energy generates, as response, an alternating magnetic field that is received via antennas.

A spatial encoding that subsequently enables the received signal to be assigned to a volume element is superimposed on the signals with the aid of magnetic gradient fields. The received signal is then evaluated, and a three-dimensional imaging visualization of the examination subject is provided. Local receive antennas, also known as local coils, may be used to receive the signals. The local coils are arranged directly on the examination subject in order to achieve a better signal-to-noise ratio.

A magnetic resonance tomography system permits a visualization of the interior of the body over a relatively long time, without exposing the patient or operator to an increased dose of ionizing radiation. On account of the low signal strengths and long integration times necessary as a consequence, it is difficult to use magnetic resonance tomography for monitoring in real time. Conventional local coils, which improve the signal acquisition, represent an obstacle during the intervention due to their necessary proximity to the region that is to be imaged.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a local coil that facilitates the monitoring of an intervention is provided.

The local coil according to the present embodiments has a central antenna coil. An antenna coil in the present context is regarded as an antenna that is configured to acquire signals of processing nuclear spins in a static magnetic field B0 of the field magnet. In one embodiment, the antenna coil is an induction loop having one or more turns that picks up a magnetic component of the magnetic resonance signal.

The central antenna coil has an opening for passing through an instrument. A surgical instrument (e.g., a needle, such as for performing a biopsy) may be guided through the opening. The opening is arranged in this case such that, when disposed on the patient over a region that is to be examined or treated, an instrument may be introduced through the opening through the skin of the patient into the region that is to be examined. In one embodiment, the opening is surrounded on an outer circumference of the opening by the central antenna coil. For example, the induction loop may enclose an area in which the opening is arranged. A ring-shaped or polygonal housing having a circular or polygonal central opening may be provided, for example. The induction loop may be arranged in the housing so that the induction loop is protected. The housing may also include a flexible material, and the induction loop may be mounted on a flexible circuit board or have a flexible cable such that the central antenna coil as a whole is flexible in order to match the surface of the patient.

The local coil according to the present embodiments has a first plurality of first peripheral antenna coils. Such a plurality in this case involves at least two, but may include three or more peripheral antenna coils.

The first peripheral antenna coils surround the central antenna coil on an outer circumference of the central antenna coil. In other words, the peripheral antenna coils are arranged adjacent to the central antenna coil such that at least two of the peripheral antenna coils are not disposed on a straight line with the central antenna coil, but vectors from a center point or center of gravity of the central antenna coil to the center points or centers of gravity of the peripheral antenna coils span a two-dimensional coordinate system. In the case of three peripheral antenna coils, for example, an equilateral triangle composed of peripheral antenna coils and enclosing the central antenna coil may be provided (e.g., in the case of four, a square; in the case of more first peripheral antenna coils, a corresponding polygon).

Adjacent may be that the central antenna coil and the first peripheral antenna coils are in contact with one another and/or may partially overlap, though do not cover one another completely. In one embodiment, the peripheral antenna coils are arranged at a short distance from the central antenna coil. A distance less than 20%, 10%, or 5% of the greatest dimension of the central antenna coil is to be regarded as short in this context.

The local coil according to the present embodiments is configured to be arranged flat on a body surface of a patient. For example, the local coil may be contoured as a substantially two-dimensional area of the surface of the body. In one embodiment, however, the local coil may be flexibly molded to fit a surface of the body. A local coil according to the present embodiments is regarded as substantially two-dimensional if its dimensions along a surface normal are less than 20%, 10%, or 5% of the dimensions along the surface.

In one embodiment, the local coil, with the aid of the central antenna coils and the peripheral antenna coils, enables fast and accurate tracking of the instrument in an extended field of view and at the same time affords good access through the opening to the region that is to be examined.

In an embodiment of the local coil, in order to realize the inductive decoupling from the first plurality of peripheral coils, the central antenna coil has a non-empty intersection of a projection of each antenna coil of the first plurality of peripheral antenna coils onto the central antenna coil along a surface normal of the central antenna coil. What is regarded as a projection of the antenna coil in this context is, for example, a projection of an area enclosed by an induction loop of the antenna coil. In other words, each of the antenna coils of the first plurality of peripheral antenna coils has an overlap with the central antenna coil. The overlap is configured in this case such that a current induced in the peripheral antenna coil by the central antenna coil by its magnetic field is precisely compensated for by the current induced by the overlap in the peripheral antenna coil as a result of the reversed sign. This conversely also applies to the field induced in the central antenna coil by the peripheral antenna coil.

The central antenna coil in this case has first sections alternating on the outer circumference of the central antenna coil along the periphery. Each of the first sections overlaps with an antenna coil of the first plurality of peripheral antenna coils. The central antenna coil also has second sections that do not overlap with an antenna coil of the first plurality of peripheral antenna coils. In other words, the central antenna coil has sections along the circumference that have no overlap with respect to an adjacent peripheral antenna coil. With regard to the concept of the overlap, reference is made in this case to the disclosure above.

In one embodiment, the overlapping sections are arranged such that the overlapping sections overlap with only one adjacent antenna coil in each case (e.g., not simultaneously with two peripheral antenna coils of the first plurality of peripheral antenna coils). This applies, for example, when the peripheral antenna coils are decoupled from one another by common conductor elements as explained below.

Using the alternating sections, the decoupling of the central antenna coil having peripheral antenna coils may be set largely independently of the decoupling of adjacent peripheral antenna coils from one another.

The first section is in this case electrically connected to the central antenna coil such that when a current flows through the central antenna coil, a magnetic field generated by the first section has an opposite polarity to a magnetic field generated by an interior space of the central antenna coil. This may be achieved, for example, in that the conductors of the induction loop crisscross at the transition to the first section such that, comparable to a figure eight, the central antenna coil divides into two sections having opposite current flow directions. The first section in this case has an overlap or an intersection with two adjacent antenna coils of the first plurality of peripheral antenna coils.

In one embodiment, sensitivity distributions comparable to a butterfly coil having a different spatial characteristic and polarity may also be achieved as a result of the opposite polarities.

In an alternative embodiment of the local coil, the central antenna coil has a first common conductor segment with an antenna coil of the first plurality of peripheral antenna coils. In one embodiment, the central antenna coil shares conductor segments with a plurality of or all of the peripheral antenna coils adjacent to the central antenna coil. In one embodiment, the entire conductor loop of the central antenna coil is formed by conductor segments of the adjacent peripheral antenna coils.

In one embodiment, a decoupling of central antenna coil and adjacent peripheral antenna coils may also be achieved by common conductor elements.

In an embodiment of the local coil, the local coil has a second plurality of peripheral antenna coils. These are substantially comparable to the first plurality of peripheral antenna coils, though the second plurality of peripheral antenna coils differ in terms of arrangement. With regard to the remaining features, the same applies as was already disclosed in relation to the first plurality of peripheral coils above. The second peripheral antenna coils surround the first peripheral antenna coils and the central antenna coil on an outer circumference of the central antenna coil. In other words, the second peripheral antenna coils are arranged adjacent to a side of the first plurality of antenna coils facing away from the central antenna coil such that the second peripheral antenna coils are at a greater distance from the central antenna coil than the first plurality of antenna coils. In one embodiment, at least two peripheral antenna coils of the second plurality of peripheral antenna coils are not located on a straight line with the central antenna coil, but vectors from a center point or center of gravity of the central antenna coil to the center points or centers of gravity of the peripheral antenna coils span a two-dimensional coordinate system.

The second plurality of peripheral antenna coils may form, for example, a polygon or a ring that encloses or surrounds the central antenna coil and the first plurality of peripheral antenna coils.

The second plurality of peripheral antenna coils may enable an instrument to be tracked in a greater volume during an intervention.

In a possible embodiment of the method, one or more of the peripheral antenna coils have an opening for passing through an instrument. This may be either an antenna coil of the first plurality of peripheral antenna coils or an antenna coil of the second plurality of peripheral antenna coils. With regard to the opening, the disclosure above in relation to the opening of the central antenna coil applies.

An opening in a peripheral antenna coil may enable a second instrument to be used simultaneously during an intervention while in the process minimizing mutual obstruction during the guidance.

In a possible embodiment of the local coil, two adjacent antenna coils of the first plurality of peripheral antenna coils have a second common conductor segment for decoupling. The remarks made in relation to the first conductor segment apply here.

In one embodiment, the decoupling of adjacent peripheral antenna coils may also be realized by conductor segments.

In an embodiment of the local coil, the first common conductor segment and/or the second common conductor segment has a decoupling element. In other words, the first conductor segment and/or the second conductor segment has an interruption that is bridged by a decoupling element. The decoupling element may be a capacitor having fixed or variable capacitance. In one embodiment, an inductor or a combination with a complex resistor is provided.

The capacitance may enable the decoupling effect of the common conductor segments to be modified given a predefined geometry.

Basically, the described decoupling variants may be combined in this case in different variants. For example, the peripheral antenna coils may be decoupled from one another by common conductor segments, and the decoupling from the central antenna coil may be realized by overlap, or vice versa.

In a possible embodiment of the local coil, the local coil has a plurality of electronics units. Devices referred to as electronics units include preamplifiers, also known as low noise amplifiers (LNAs), matching networks, and tuning or detuning circuits, as well as combinations thereof. The electronics unit may be realized, for example, on a printed circuit on a substrate or flexible circuit board. In one embodiment, the flexible circuit board provides the signal connection or even the antenna coil itself. The electronics units are connected to the central antenna coil and the first plurality of peripheral antenna coils for signal communication purposes. In one embodiment, each antenna coil is assigned a separate electronics unit. In one embodiment, two or three antenna coils in each case share a common electronics unit and are connected to the common electronics unit. An electronics unit connected to a peripheral antenna coil for signal communication purposes is in this case arranged directly outside on a side of the respective peripheral antenna coil facing away from the central antenna coil. Considered as direct in this context is a distance that keeps the length of the signal connection or cable between antenna coil and electronics unit as short as possible (e.g., less than a diameter of the antenna coil, less than twice the diameter of the antenna coil, or 50% of the diameter).

### BRIEF DESCRIPTION OF THE DRAWINGS

FIG. 1 shows a schematic representation of a magnetic resonance tomography system having a local coil according to an embodiment;

FIG. 2 shows a schematic representation of an embodiment of the local coil;

FIG. 3 shows a schematic representation of an embodiment of the local coil;

FIG. 4 shows a schematic representation of an embodiment of the local coil;

FIG. 5 shows a schematic representation of an embodiment of the local coil;

FIG. 6 shows a schematic representation of an embodiment of the local coil; and

FIG. 7 shows a schematic representation of an embodiment of the local coil.

### DETAILED DESCRIPTION

FIG. 1 shows a schematic representation of an embodiment of a magnetic resonance tomography system 1 for performing a method according to the present embodiments.

A magnet unit 10 has a field magnet 11 that generates a static magnetic field B0 for aligning nuclear spins of specimens or of a patient 100 in an acquisition region. The acquisition region is characterized by an extremely homogeneous static magnetic field B0. The homogeneity relates, for example, to the magnetic field strength or the absolute value. The acquisition region is virtually spherical in shape and is arranged in a patient tunnel 16 that extends in a longitudinal direction 2 through the magnet unit 10. A patient couch 30 may be moved in the patient tunnel 16 by the positioning unit 36. Typically, the field magnet 11 is a superconducting magnet that is able to provide magnetic fields having a magnetic flux density of up to 3 T, even higher in the case of the latest devices. For lower magnetic field strengths, however, permanent magnets or electromagnets with normally conducting coils may also find application.

In addition, the magnet unit 10 has gradient coils 12 that are configured to overlay the magnetic field B0 with temporally and spatially variable magnetic fields in three spatial directions in order to spatially differentiate the acquired imaging regions in the examination volume. The gradient coils 12 are typically coils composed of normally conducting wires that may generate fields orthogonal to one another in the examination volume.

The magnet unit 10 also has a body coil 14 that is configured to radiate a radiofrequency signal supplied via a signal line into the examination volume and to receive resonance signals emitted from the patient 100 and pass the received resonance signals on via a signal line.

A control unit 20 supplies the magnet unit 10 with the different signals for the gradient coils 12 and the body coil 14 and evaluates the received signals.

Thus, the control unit 20 has a gradient controller 21 that is configured to supply the gradient coils 12 via feeder lines with variable currents that provide the desired gradient fields in the examination volume in a coordinated manner with respect to time.

The control unit 20 also has a radiofrequency unit 22 that is configured to generate a radiofrequency pulse having a predefined time characteristic, amplitude, and spectral power distribution in order to excite a magnetic resonance of the nuclear spins in the patient 100. Pulse powers in the kilowatt range may be achieved in this case. The excitation signals may be radiated into the patient 100 via the body coil 14 or also via a local transmit antenna.

A controller 23 communicates with the gradient controller 21 and the radiofrequency unit 22 via a signal bus 25.

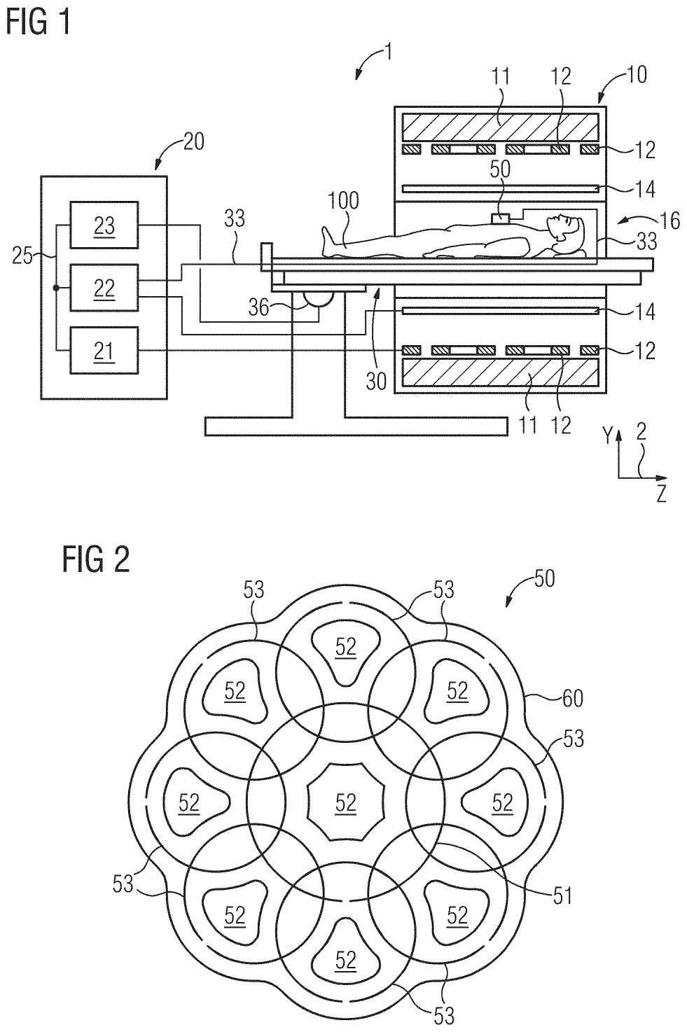

FIG. 2 shows a schematic representation of an embodiment of the local coil 50 according to the present embodiments. For clarity of illustration reasons, the electrical signal connections of the local coil 50 are not shown in FIG. 2.

The local coil 50 shown in FIG. 2 has a central antenna coil 51. The central antenna coil 51 surrounds a central opening 52 through which an instrument may be guided for performing an examination or an intervention. The central antenna coil 51 may in this case have one or more turns or loops of an electrical conductor. The conductor may be implemented as a wire or coaxial cable, for example. A flexible carrier material on which a conductor track is deposited may also be provided.

The central antenna coil 51 is surrounded by a plurality of peripheral antenna coils 53 arranged in a ring shape or in the form of an octagon. With regard to the peripheral antenna coils 53, the disclosure in relation to the central antenna coil 51 applies with respect to the embodiment of the conductor loop. In one embodiment, the central antenna coil 51 and the peripheral antenna coils 53 are implemented similarly, though the central antenna coil 51 and the peripheral antenna coils 53 may also differ in terms of material, dimensions, electrical properties, and/or shape. Examples of this are presented in relation to the following figures.

The central antenna coil 51 and the peripheral antenna coil 53 as well as the peripheral antenna coils 53 among one another have sections at which areas enclosed by the respective antenna coils 51, 53 overlap. Inside the overlapping section, an antenna coil 51, 53 generates a magnetic field that has a reverse polarity to the magnetic field in a region outside of the antenna coil 51, 53 and consequently, given a suitable choice of the area of the overlapping section, the reciprocally induced currents cancel one another out. This leads to a decoupling of the antenna coils 51, 53.

The peripheral antenna coils 53 may also have openings through which a second instrument may be guided.

In one embodiment, the central antenna coil 51 and the peripheral antenna coils 53 are surrounded by a housing 60 of the local coil 50 in order to protect the antenna coils 51, 53 against soiling and at the same time to provide that the patient 100 is not put at risk due to touching a conductor of the antenna coils 51, 53. The housing 60 in this case has openings corresponding to the openings 52 of the central antenna coil 51 and the peripheral antenna coils 53 such that an instrument may be guided through the central antenna coil 51 and/or peripheral antenna coil 53 into or onto the patient 100.

The local coil 50 is configured to be arranged flat on a body surface of a patient. For example, the local coil 50 may be embodied as a planar element. In one embodiment, the local coil 50 and a housing 60 of the local coil 50 are implemented as flexible so that the housing 60 may be matched to the body shape. In one embodiment, however, the local coil 50 is contoured to match a part of the body.

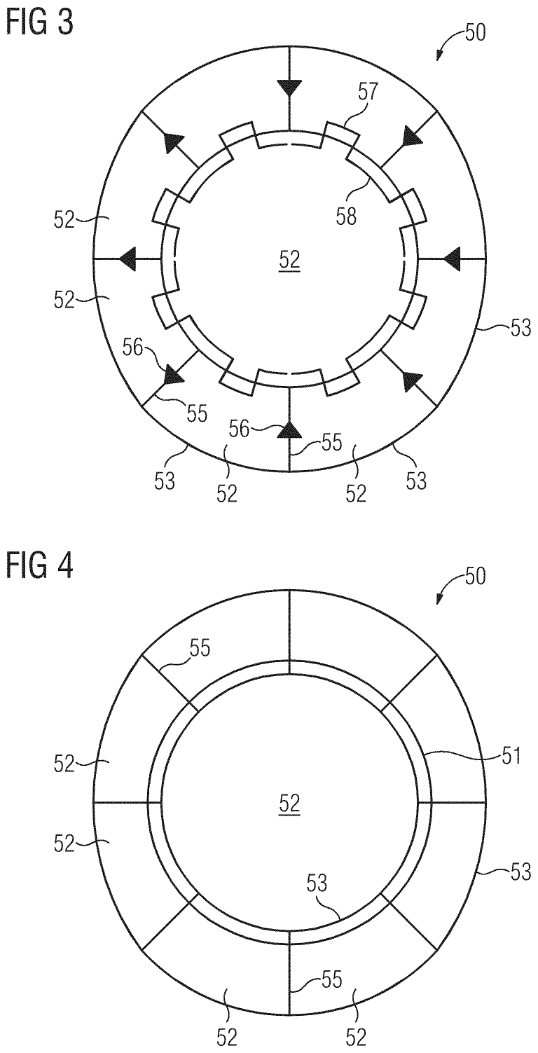

FIG. 3 shows a further embodiment of the local coil 50. Like elements are labeled with like reference signs. For clarity of illustration reasons, the housing 60 is not shown in FIG. 3. said the disclosure in relation to the housing 60 with reference to FIG. 2 applies analogously.

In FIG. 3, the peripheral antenna coils 53 are embodied, not as circular antenna coils, but as segments of a ring around the central antenna coil 51. In this case, adjacent peripheral antenna coils 53 are not ohmically insulated from one another but have second common conductor elements 55. The second common conductor elements 55 provide the decoupling of the adjacent peripheral antenna coils 53 and replace the overlap of the adjacent peripheral antenna coils 53 from FIG. 2. In this arrangement, the second common conductor element 55 may have an interruption that is bridged by a complex impedance. The complex impedance enables the decoupling to be adjusted or optimized.

The decoupling between central antenna coil 51 and peripheral antenna coils 53 is realized in FIG. 3 by an overlap of the central antenna coil 51 with the respective peripheral antenna coils 53. In this case, however, first sections 57, in which an overlap with an adjacent peripheral antenna coil 53 is present, are arranged along the circumference of the central antenna coil 51 alternating with second sections 58 at which no overlap is present. In an embodiment, the first sections 57 are arranged such that no overlap with a second common conductor element is present. Using the dimensioning of the first sections 57, it is possible to achieve an optimal decoupling of the central antenna coil 51 from the peripheral antenna coils 53 that has only a minor interaction with the reciprocal decoupling of the peripheral antenna coils 53 from one another.

Although the housing 60 is not shown, both the central antenna coil 51 and the peripheral antenna coils 53 have an opening 52 in each case. The openings 52 are likewise embodied in the housing 60 and thus allow access by an instrument through the central antenna coil 51 and the peripheral antenna coils 53.

FIG. 4 shows a further embodiment of the local coil 50. In the embodiment of FIG. 4, in contrast to FIG. 3, the decoupling between central antenna coil 51 and peripheral antenna coil 53 is realized by a continuous overlap along the circumference of the peripheral antenna coil 53. As in FIG. 3, decoupling elements may be provided at the second common conductor elements 55.

FIG. 5 schematically shows a further embodiment of the local coil. In FIG. 5, the decoupling is now realized only by common conductor elements 54, 55. This may even lead, as in FIG. 5, to the central antenna coil 51 now being formed only of first common conductor segments 54. It is also possible, however, that first common conductor segments having the peripheral antenna coils 53 alternate along the circumference of the central antenna coil with conductor segments belonging only to the central antenna coil 51.

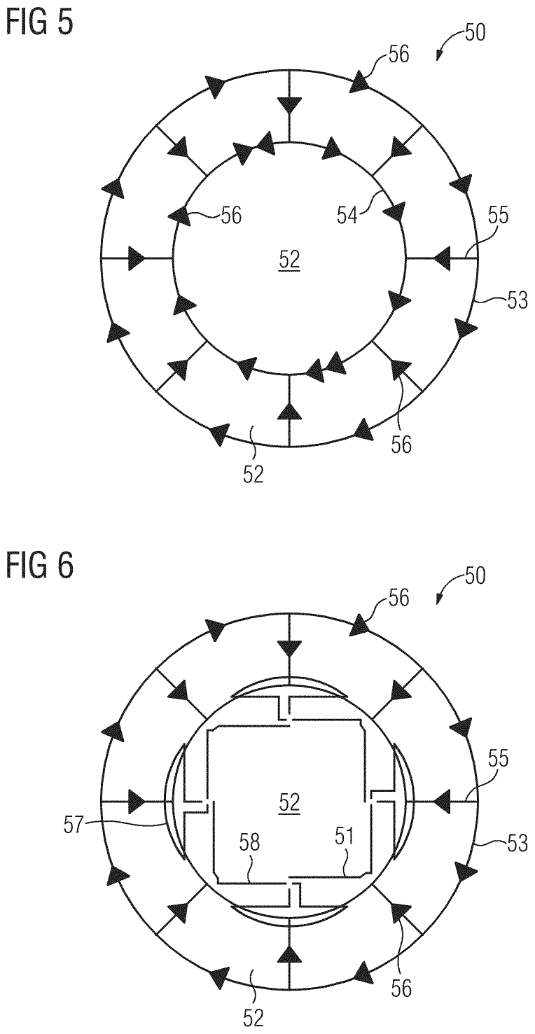

FIG. 6 schematically shows a further embodiment of the local coil. As in FIG. 3, the peripheral antenna coils 53 are decoupled from the central antenna coil 51 by alternating overlapping and non-overlapping sections along the circumference of the central antenna coil 51. In contrast to the embodiment of FIG. 3, the overlapping first section 57 is arranged precisely such that the overlapping first section 57 simultaneously overlaps with two peripheral antenna coils 53 adjacent to each other. In order to minimize the interaction of the three antenna coils, in this case, in contrast to the first section in FIG. 3, the overlap is connected by crisscrossing connection lines to the central antenna loop such that this has an opposite current circulation direction. As a result of this, the overlapping first section generates a magnetic field having the same sign as in the interior space of the central antenna coil.

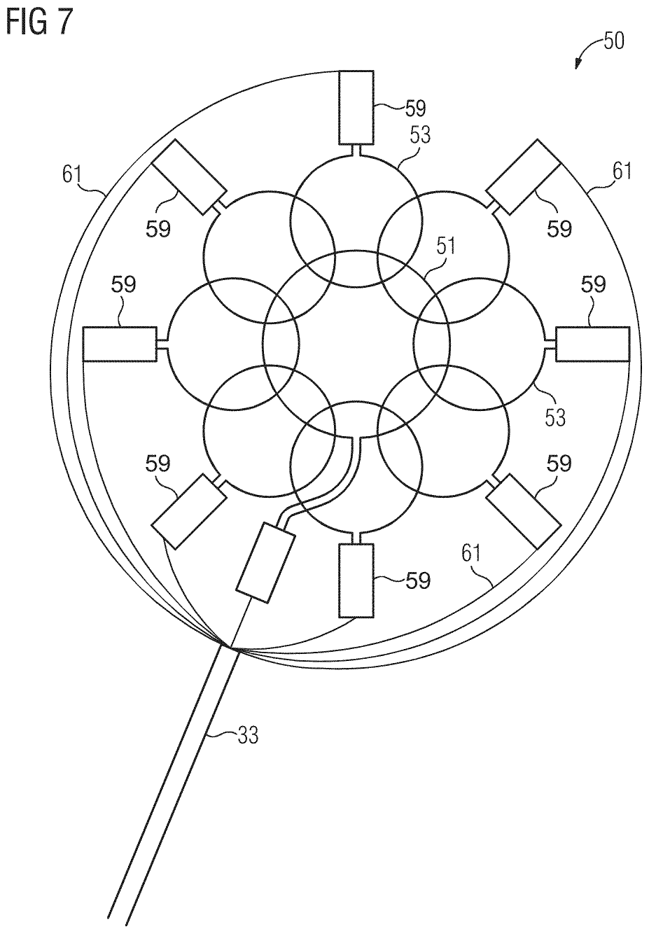

FIG. 7 shows an embodiment for forwarding the magnetic resonance signals acquired by the antenna coils 51, 53 to the magnetic resonance tomography system based on the example of the local coil 50 from FIG. 2. This may also find application as such or in variations for the embodiments shown in FIGS. 3 to 6.

The local coil 50 has a plurality of electronics units 59. An electronics unit of the plurality of electronics units 59 may have one or more of the following functional units: low-noise amplifier (LNA); matching circuit for matching coils to cable or amplifier impedance; active and/or passive detuning circuits for detuning the antenna coil during the transmission of the excitation pulse; and cutouts in the event of failure of the detuning circuits.

The electronics units 59 in each case maintain a signal connection to the central antenna coil 51 and the first plurality of peripheral antenna coils 53. In one embodiment, this is an electrical connection. The signal connection 61 forwards the received magnetic resonance signal from the antenna coil 51, 53 to the electronics unit 59.

In an embodiment, the arrangement of the electronics units 59 relative to the peripheral antenna coils 53 is chosen such that the signal connection 61 between the antenna coils 51, 53 and the electronics unit 59 is as short as possible but at the same time does not obstruct access through the openings 62 to the patient 100 and/or reduce the image quality. In one embodiment, one electronics unit 59 having a signal connection to a peripheral antenna coil 53 is arranged directly outside on a side of the respective peripheral antenna coil 53 facing away from the central antenna coil 51. In one embodiment, one electronics unit 59 supplies two adjacent peripheral antenna coils 53 and is arranged centrally between the two peripheral antenna coils 53 on the facing-away side of the two peripheral antenna coils 53, such that the signal connection to the two peripheral antenna coils 53 is as short as possible.

In one embodiment, the signal connections 61 are dimensioned longer, for example, when the functions of the electronics unit 59 for a plurality of or all the antenna coils 51, 53 are centralized in one assembly.

Output signals of the electronics units 59 are bundled in a connection line 33 and forwarded to the magnetic resonance tomography system 1. In the reverse direction, the electronics units 59 receive the energy and the control signals (e.g., for the detuning circuits) from the magnetic resonance tomography system 1.

In one embodiment, a wireless local coil 50 according to the present embodiments manages without a connection line 33. In this case, the local coil still has its own energy supply (e.g., in the form of a rechargeable battery), as well as a wireless transmission facility (e.g., via a WLAN technology or optically).

Although the invention has been illustrated and described in more detail based the exemplary embodiments, the invention is not limited by the disclosed examples, and other variations may be derived herefrom by the person skilled in the art without leaving the scope of protection of the invention.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A local coil for percutaneous magnetic resonance tomography (MRT)-guided minimally invasive intervention, the local coil comprising:
a central antenna coil having an opening for passing through an instrument; and
a plurality of first peripheral antenna coils, wherein the plurality of first peripheral antenna coils surround the central antenna coil on an outer circumference of the central antenna coil,
wherein the local coil is configured to be arranged flat on a body surface of a patient,
wherein in order to realize an inductive decoupling from the plurality of first peripheral coils, the central antenna coil has a non-empty intersection of a projection of each antenna coil of the plurality of first peripheral antenna coils onto the central antenna coil along a surface normal of the central antenna coil,
wherein the central antenna coil has first sections on the outer circumference alternating along a periphery, each of which overlaps with an antenna coil of the plurality of first peripheral antenna coils, and second sections that do not overlap with an antenna coil of the plurality of first peripheral antenna coils, and
wherein a first section of the first sections is electrically connected to the central antenna coil such that when a current flows through the central antenna coil, a magnetic field generated by the first section has an opposite polarity to a magnetic field generated by an interior space of the central antenna coil, and the first section has an overlap with two adjacent antenna coils of the plurality of first peripheral antenna coils.

2. The local coil of claim 1, further comprising a plurality of second peripheral antenna coils, wherein the plurality of second peripheral antenna coils surround the plurality of first peripheral antenna coils and the central antenna coil on the outer circumference of the central antenna coil.

3. The local coil of claim 1, wherein one or more peripheral antenna coils of the plurality of first peripheral antenna coils have an opening for passing through an instrument.

4. The local coil of claim 1, wherein in order to realize an inductive decoupling from an adjacent peripheral antenna coil of the plurality of first peripheral antenna coils, a peripheral antenna coil of the plurality of first peripheral antenna coils has a non-empty intersection of a projection of the peripheral antenna coil onto the adjacent peripheral antenna coil along a surface normal of the peripheral antenna coil.

5. The local coil of claim 1, further comprising a plurality of electronics units that are in signal connection with the central antenna coil, the plurality of first peripheral antenna coils, or the central antenna coil and the plurality of first peripheral antenna coils,
wherein one electronics unit of the plurality of electronics units that is in signal connection with a peripheral antenna coil of the plurality of first peripheral antenna coils is arranged directly outside on a side of the peripheral antenna coil facing away from the central antenna coil.

6. A local coil for percutaneous magnetic resonance tomography (MRT)-guided minimally invasive intervention, the local coil comprising:
a central antenna coil having an opening for passing through an instrument; and
a plurality of first peripheral antenna coils that surround the central antenna coil on an outer circumference of the central antenna coil,
wherein the local coil is configured to be arranged flat on a body surface of a patient, and
wherein the central antenna coil has a first common conductor segment with an antenna coil of the plurality of first peripheral antenna coils.

7. The local coil of claim 6, further comprising a plurality of second peripheral antenna coils, wherein the plurality of second peripheral antenna coils surround the plurality of first peripheral antenna coils and the central antenna coil on the outer circumference of the central antenna coil.

8. The local coil of claim 6, wherein one or more peripheral antenna coils of the plurality of first peripheral antenna coils have an opening for passing through an instrument.

9. The local coil of claim 6, wherein in order to realize an inductive decoupling from an adjacent peripheral antenna coil of the plurality of first peripheral antenna coils, a peripheral antenna coil of the plurality of first peripheral antenna coils has a non-empty intersection of a projection of the peripheral antenna coil onto the adjacent peripheral antenna coil along a surface normal of the peripheral antenna coil.

10. The local coil of claim 6, wherein two adjacent antenna coils of the plurality of first peripheral antenna coils have a second common conductor segment for the decoupling.

11. The local coil claim 10, wherein the first common conductor segment, the second common conductor segment, or the first common conductor segment and the second common conductor segment have a decoupling element.

12. The local coil of claim 6, further comprising a plurality of electronics units that are in signal connection with the central antenna coil, the plurality of first peripheral antenna coils, or the central antenna coil and the plurality of first peripheral antenna coils,
wherein one electronics unit of the plurality of electronics units that is in signal connection with a peripheral antenna coil of the plurality of first peripheral antenna coils is arranged directly outside on a side of the peripheral antenna coil facing away from the central antenna coil.
